(19) United States
(12) Patent Application Publication
(10) Pub. No.: US 2025/0152802 A1
(43) Pub. Date: May 15, 2025

(54) MEDICAL DEVICES WITH A NEGATIVE PRESSURE SOURCE AND RELATED SYSTEMS AND METHODS OF USE

(71) Applicant: Boston Scientific Scimed, Inc., Maple Grove, MN (US)

(72) Inventors: Paul SMITH, Smithfield, RI (US); Jason WEINER, Grafton, MA (US); Martyn FOLAN, Loughrea, Co. Galway (IE); Brittney L. SEIBERT, San Diego, CA (US)

(73) Assignee: Boston Scientific Scimed, Inc., Maple Grove, MN (US)

(21) Appl. No.: 18/945,758

(22) Filed: Nov. 13, 2024

Related U.S. Application Data
(60) Provisional application No. 63/599,056, filed on Nov. 15, 2023.

(51) Int. Cl. A61M 1/00

(52) U.S. Cl. CPC A61M 1/962; A61M 1/79

(57) ABSTRACT

According to one aspect, a medical device system may comprise a bag including a first opening; a pad coupled to the bag proximate to the first opening; and a tube including a distal portion. The distal portion may be coupled to the pad and includes at least one opening; and the tube may be configured to provide negative pressure therapy to a wound site.

### CROSS-REFERENCE TO RELATED APPLICATIONS

This application claims the benefit of priority of U.S. Provisional Patent Application No. 63/599,056, filed on Nov. 15, 2023, the entirety of which is incorporated herein by reference.

### TECHNICAL FIELD

Various aspects of this disclosure relate generally to systems for coupling a medical device to a body, systems for providing negative pressure therapy, and related devices and methods. In particular, aspects of this disclosure relate to devices, systems, and methods for coupling an ostomy bag to a body, among other aspects.

### BACKGROUND

In connection with surgery for a number of diseases in the gastro-intestinal tract, one of the consequences in many cases is that the patient is left with an abdominal stoma, such as a colostomy or an ileostomy in the abdominal wall for the discharge of visceral contents. The discharge of visceral contents, including intestinal gases, cannot be regulated at will. For that purpose, the user will have to rely on a device to collect the material emerging from such opening in a bag, such as an ostomy bag, which is later emptied and/or discarded at a suitable time. An ostomy is where the colon, ileum of the small intestine, or the large bowel is removed, bypassed, resected, and/or ported to the outside of the body. The site of the ostomy is often subject to infection or other complications, such as suture line break down, an infected stoma site, peristomal fistula, skin irritation, pressure ulcers, muco separation, and/or skin breakdown.

The current disclosure may solve one or more of these issues or other issues in the art.

### SUMMARY

Embodiments of this disclosure relate to, among other things, medical devices and systems for treating a patient and/or coupling a medical device to a patient, and related methods of use. Embodiments of this disclosure may facilitate healing at a wound site, may enhance the coupling of a medical device to a patient, and/or may help to reduce infection at a wound site. Each of the embodiments disclosed herein may include one or more of the features described in connection with any of the other disclosed embodiments.

According to one aspect, a medical device system may comprise a bag including a first opening; a pad coupled to the bag proximate to the first opening; and a tube including a distal portion. The distal portion may be coupled to the pad and includes at least one opening; and the tube may be configured to provide negative pressure therapy to a wound site.

In other aspects, the medical device system may include one or more of the following features. A pump assembly may be fluidically coupled to the tube, and the pump assembly may apply negative pressure to the tube. The pad may be circular and include a central lumen, and the central lumen may align with the first opening. The distal portion of the tube may extend circumferentially around the central lumen. The at least one opening of the tube may include a plurality of openings, and the plurality of openings may face a direction towards the pad. A filter assembly may be coupled to the tube and configured to filter material received from the wound site. The pump assembly may include a pump and a reservoir configured to receive material from a wound site. The pad may include a channel configured to receive the tube, and the channel may extend circumferentially around a central lumen of the pad. The pad may include a perforated portion configured to be removable from the pad. The pad may include a first layer of porous material and a second layer of porous material, and a portion of the tube may be positioned between the first layer of porous material and the second layer of porous material. The pad may include an adhesive on a first side of the pad, and the adhesive may extend across a first portion of the first side of the pad and not across a second portion of the first side of the pad. The medical device system may further comprise a wafer, and a first side of the wafer may be configured to couple the bag to the wafer, and a second side of the wafer may be configured to couple the pad to the wafer. The wafer may include a convex portion, and the convex portion may extend circumferentially around a central lumen of the wafer, and the tube may be positioned radially-outward, relative to a central longitudinal axis of the central lumen, relative to the convex portion. The bag may include a pocket and the pump assembly may be positioned within the pocket. The pad may be C-shaped.

In other aspects, a medical device system may comprise a pad configured to couple to tissue, wherein the pad includes a central lumen; a first tube including a first distal portion, wherein the first distal portion is coupled to the pad, extends circumferentially around the central lumen, and includes at least one opening; and a second tube including a second distal portion, wherein the second distal portion is coupled to the pad, extends circumferentially around the central lumen, and includes at least one opening. The first tube is configured to provide negative pressure therapy to a wound site; and the second tube is configured to facilitate coupling the pad to the tissue.

In other aspects, the medical device system may include one or more of the following features. The first distal portion may include a plurality of openings facing towards the central lumen, and the second distal portion may include a plurality of openings facing away from the central lumen. The first tube may be configured to apply a first negative pressure, the second tube may be configured to apply a second negative pressure, and the first negative pressure may be different from the second negative pressure. The medical device system may further comprise an ostomy bag, wherein the pad is coupled to the ostomy bag.

In other aspects, a medical device system may comprise a pad configured to couple to tissue, wherein the pad is a porous material; a tube including a distal portion, wherein the distal portion is coupled to the pad and includes a plurality of openings facing towards the pad; and a pump assembly coupled to a proximal portion of the tube. The pump assembly may be configured to provide negative pressure to the tube, the medical device system may be configured to provide negative pressure therapy to a wound site, and the tube may be positioned within a channel extending circumferentially around a central lumen of the pad.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Although ostomy bags are referenced herein, reference to ostomy bags should not be construed as limiting the possible applications of the disclosed aspects. For example, the disclosed aspects may be used with wound dressings such as band aids, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 is a perspective view of a medical device system coupled to a patient, according to aspects of this disclosure.

FIG. 2 is a perspective view of a portion of a medical device, according to aspects of this disclosure.

FIG. 3 is a perspective view of a disassembled medical device, according to aspects of this disclosure.

FIG. 4 is a perspective view of portions of a medical device system, according to aspects of this disclosure.

FIGS. 5A-5D illustrate perspective views of assembling portions of a medical device system, according to aspects of this disclosure.

FIG. 6 illustrates perspective and side cross-sectional views of portions of a medical device system, according to aspects of this disclosure.

FIG. 7 shows a top view of a distal tip section of a medical device, according to aspects of this disclosure.

FIG. 8 shows a side view of a portion of a medical device, according to aspects of this disclosure.

FIG. 9 shows a side view of a portion of a medical device, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

Embodiments of this disclosure seek to improve coupling a medical device to patient, reducing infection at a wound site, reducing pain and complications at a wound site, facilitating healing at a wound site, and/or facilitating draining fluids from a patient. Embodiments of this disclosure seek to improve ostomy bags and related devices, and improve therapy treatments using negative fluid pressure.

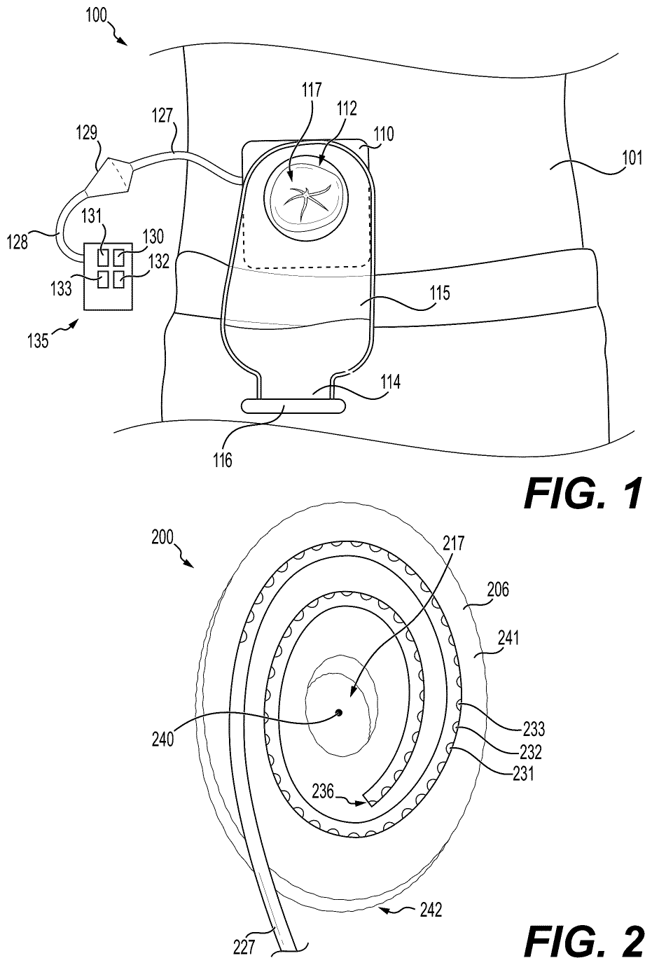

An exemplary medical device system 100 is shown in FIG. 1. Medical device system 100 may include a bag 115, a wafer 110, a pad 112, one or more tubes 127, 128, and a pump assembly 135. Bag 115 may be an ostomy bag, and may include a distal opening 117 and a proximal opening 114 covered by a proximal end cap 116. Bag 115 may be coupled to a patient 101 via wafer 110 and pad 112. For example, bag 115 may be coupled to a patient's abdomen and may be configured to receive material and/or fluids from the digestive system of the patient 101, such as when the small intestine or large intestine is resected. Proximal end cap 116 may provide a means for the patient to empty bag 115, by removing proximal end cap 116 and disposing of material collected in bag 115. Bag 115 may be plastic or any other suitable material.

Bag 115 may be directly coupled to wafer 110, pad 112, or both wafer 110 and pad 112. Wafer 110 and/or pad 112 may include one or more adhesives configured to couple to skin of patient 101, such as skin around a wound site of patient 101. Each of wafer 110 and pad 112 may include a central lumen, such as central lumen 217 (shown in FIG. 2) configured to align with a wound/resection site of patient 101. Tube 127 may be coupled to pad 112 and/or wafer 110, and tube 127 may be configured to provide negative pressure therapy to patient 101 proximate to a wound/resection site. One or more tubes 127, 128 may be operatively coupled to pump assembly 135, and pump assembly 135 may apply negative fluid pressure to one or more tubes 127, 128. In some examples, a filter assembly 129 may be coupled to one or more tubes 127, 128 between pump assembly 135 and wafer 110 and/or pad 112.

Pump assembly 135 may include a pump, a reservoir for receiving material from one or more tubes 127, 128, and one or more actuators 130-133. Pump assembly 135 may also include any suitable connectors (e.g., tubing for fluidly coupling components of pump assembly). Pump assembly 135 may be configured to supply negative fluid pressure to one or more tubes 127, 128. Tubes 127, 128 may be releasably coupled to pump assembly 135 such that a user may connect and disconnect one or more tubes 127, 128 from pump assembly 135. Pump assembly 135 may receive material from patient 101 collected by the negative pressure supplied to one or more tubes 127, 128, for example, to facilitate wound healing and assist with coupling bag 115 to patient 101. In some examples, pump assembly 135 may generate from approximately 60 mmhg pressure to approximately 130 mmhg pressure (inclusive), and a user may select any pressure within that range to be applied to one or more tubes 127, 128. For example, pump assembly 135 may apply 125 mmhg of negative pressure to one or more tubes 127, 128. In some examples, pump assembly 135 may be preset to start at a given desired pressure, such as 125 mmhg. Reservoir of the pump assembly 135 may be a canister or any other suitable container for collecting material. Filter assembly 129 may be configured to trap and/or filter solid and/or semi-solid material and prevent such material from entering into pump assembly 135.

In some examples, pump assembly 135 may be configured to be portable (e.g. lightweight and sized to fit and be carried by a patient 101). For example, bag 115 may include a pump assembly 135 carrying means such as a pouch, strap, clip, hook, and/or loop to maintain a proximity to bag 115 and provide mobility to a patient performing negative pressure (i.e., vacuum) therapy. As will be discussed in further detail hereinbelow, negative pressure supplied from pump assembly 135 to one or more tubes 127, 128 may provide suction to pad 112 and/or wafer 110 to treat patient 101 and/or facilitating coupling bag 115 to patient 101.

FIG. 2 illustrates an exemplary portion 200 of medical device system 100 including pad 206 and tube 227. As shown in FIG. 2, pad 206 may include a central lumen 217, and a central axis 240 may extend through central lumen 217 (shown extending through the page in FIG. 2.) Pad 206 may be made of open cell foam and/or any porous material configured to allow fluid flow through the material. In some examples, pad 206 may be a layered dressing with channels to provide a fluidic path to a patient's tissue. Pad 206 may include a layered dressing with distinct layers to provide fluid flow, absorbent material, storage of removed fluid and waste, a drying layer, and/or a layer or coating to provide antimicrobial treatment, such as silver. Tube 227 may be coupled to a first side 241 of pad 206 (shown facing out of the page), and an adhesive layer may be on a portion (or all of) a second side 242 (shown facing into the page) facing a direction opposite from the direction the first side 241 is facing.

Tube 227 may be coupled to pad 206, such as by adhesive or any other coupling means known in the art. A distal portion of tube 227 may spiral or wrap around central axis 240, and may only be coupled to first side 241 of pad 206. A proximal portion (not shown) of tube 227 may be coupled to pump assembly 135. Tube 227 may include one or more openings 231-233 on a radially-outward facing surface of tube 227, relative to a longitudinal axis of tube 227, and may also include a distal opening 236. In some examples, tube 227 may not include distal opening 236, and may include a closed distal end. In some examples, each of openings 231-233 may face towards first side 241 of pad 206. In some examples (not shown), tube 227 may be arrange in a singular ring pattern, a wave pattern, a series of connected annular rings, a C-shape, or any other suitable configuration on first side 241 of pad 206. Pad 206 may be configured to couple to a patient's 101 tissue, and tube 227 may be configured to apply negative pressure to pad 206. Thus, tube 227 may provide a suction force on patient's tissue to pull material through pad 206 to tube 227 via negative pressure applied to one or more openings 231-233, 236.

During use, portion 200 may be coupled to wafer 110, and then pad 206 may be coupled to a patient 101 at a wound/resection site. In other examples, medical device system 100 may not include wafer 110, and pad 206 may be directly coupled to bag 115. Bag 115, either before or after portion 200 is coupled to patient 101, may be coupled to wafer 110 and/or pad 206. Once coupled to a patient 101, negative pressure may be applied to one or more tubes 127, 128, 227 to supply negative pressure to openings 231, 232, 233 of tube 227. The supplied negative pressure may exert a suction force on tissue of the patient 101 at and/or proximate to the wound site. Material or fluid from patient's 101 tissue may be pulled through pad 206 and into tube 227, and collected by pump assembly 135. The negative pressure supplied to tube 207 at the wound site may facilitate tissue healing, may help to reduce the risk of contamination of the wound site, may help prevent infection, may facilitate coupling bag 115 to patient 101, and/or may collect waste and/or other material from the patient's digestive tract. A patient 101 or other user may adjust the negative pressure applied to tube 207 by actuating pump assembly 135, for example, to increase or decrease the negative fluid pressure applied at the wound site.

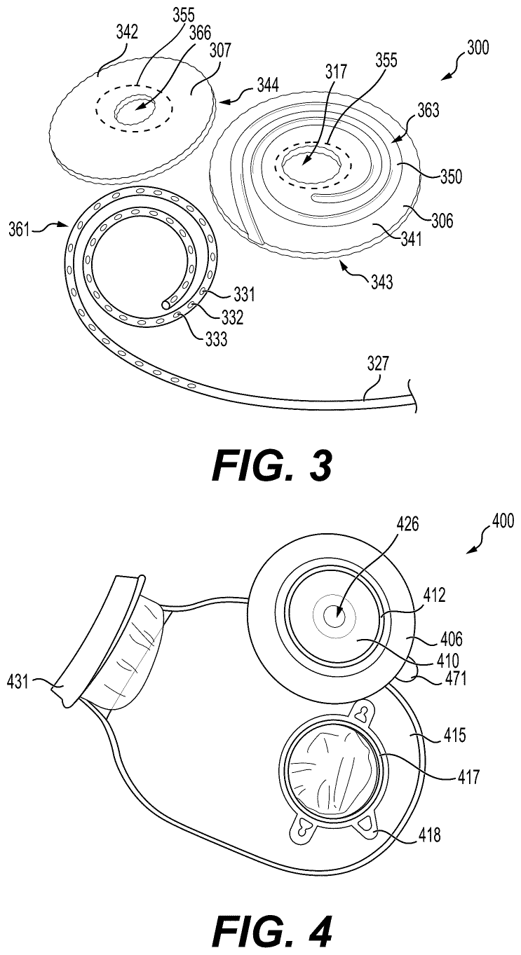

FIG. 3 illustrates another exemplary disassembled portion 300 of a medical device system (e.g., medical device system 100) including pads 306, 307 and tube 327. First pad 306 may be an absorbent material, and may include a central lumen 317 and a channel 350 extending through a first side 341 of first pad 306. Channel 350 may be configured to receive tube 327, and channel 350 may extend around central lumen 317. Channel 350 may have a depth (into first pad 306) equal to or greater than the diameter of tube 327. In some examples, channel 350 may be cut into or otherwise formed in first pad 306 in a manner to preserve the opening in first pad 306. For example, first pad 306 may be cut by mechanical means or by high heat to vaporize/pyrolysis portions of first pad 306 so not to close the openings in first pad 306. A second side 343 of first pad 306 may include an adhesive, and may be configured to couple to wafer 110 and/or bag 115. In some examples, a distal portion 361 of tube 327 may be fixedly coupled within channel 350, and one or more openings 331-333 of tube 327 may face towards an opening 363 of channel 350. Although channel 350 is shown in a spiral shape, channel 350 may have any suitable shape such as a wave pattern, connected concentric annular rings, or any other shape.

Second pad 307 may be configured to couple to and at least partially align with first pad 306. Second pad 307 may include a first side 342, a second side 344, and a central lumen 366. First side 342 of second pad 307 may include an adhesive configured to couple to tissue of patient 101 (FIG. 1), and second side 344 may include an adhesive configured to couple to first pad 306. In an assembled configuration, second pad 307 may be positioned over first pad 306 such that tube 327 is positioned within channel 350 and is positioned between first pad 306 and second pad 307. Second pad 307 may be porous and configured to allow fluid flow through second pad 307 to tube 327. In some examples, first pad 306 and/or second pad 307 may include one or more perforated portions 355, and perforated portions 355 may be configured to allow a user to remove a portion of first pad 306 and/or second pad 307, for example, to re-size central lumen 317, 366. Perforated portions 355 may allow a user to adjust the size of pads 306, 307 to accommodate different size wounds/resections.

FIG. 4 illustrates an exemplary portion 400 of a medical device system (e.g., medical device system 100) including bag 415, wafer 410, and pad 406. Bag 415 is shown with a coupling frame 418 around first opening 417 of bag 415. Bag 415 is also shown with a cap 431 over a second opening of bag 415. A ridge 412 of wafer 410 may be received by coupling frame 418 of bag to couple bag 415 to wafer 410. In some examples, wafer 410 may be screwed into coupling frame 418 via one or more threads of wafer 410 and/or coupling frame 418. Central lumen 426 of pad 406 and wafer 410 may be configured to at least partially align with a wound site of a patient 101 (FIG. 1) to receive waste and/or other material or fluid from patient 101. In some examples, pad 406 may include a removable protective cover 471 over the side of pad 406 configured to couple to tissue of patient 101. Cover 471 may include a tab that extends radially outward/outside of pad 406. A user may remove protective cover 471 to expose adhesive on pad 406 and allow user to push adhesive of pad 406 onto tissue of patient 101, in order to couple pad 406 to patient 101.

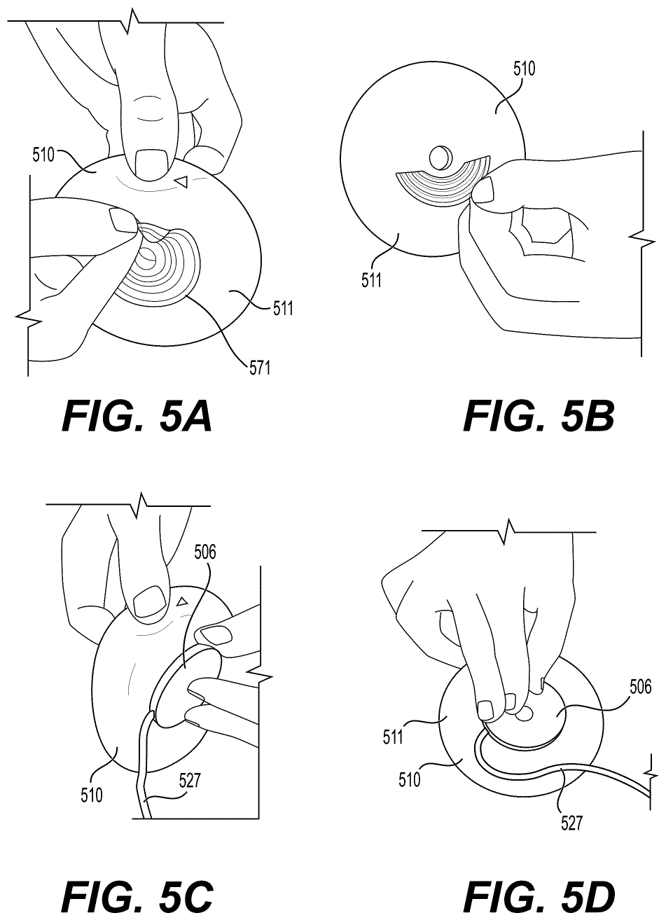

FIGS. 5A-5D illustrate a method of coupling a disk-shaped vacuum therapy pad 506 to a wafer 510 of an ostomy bag. FIGS. 5A and 5B show a user peeling off a protective covering 571 to expose an adhesive present on a first side 511 of wafer 510. FIG. 5C shows a user pushing or otherwise urging pad 506 onto the exposed adhesive of wafer 510, with tube 527 extending from pad 506. FIG. 5D shows a user pushing or otherwise urging pad 506 onto the first side 511 of wafer 510 to fixedly couple pad 506 to wafer 510. Once pad 506 is coupled to wafer 510, the user may couple an ostomy bag (not shown) to wafer 510 and proceed to couple pad 506 to patient 101. As mentioned above, pad 506 may absorb material and/or fluid from the wound site on patient 101, and negative pressure may be applied via tube 571.

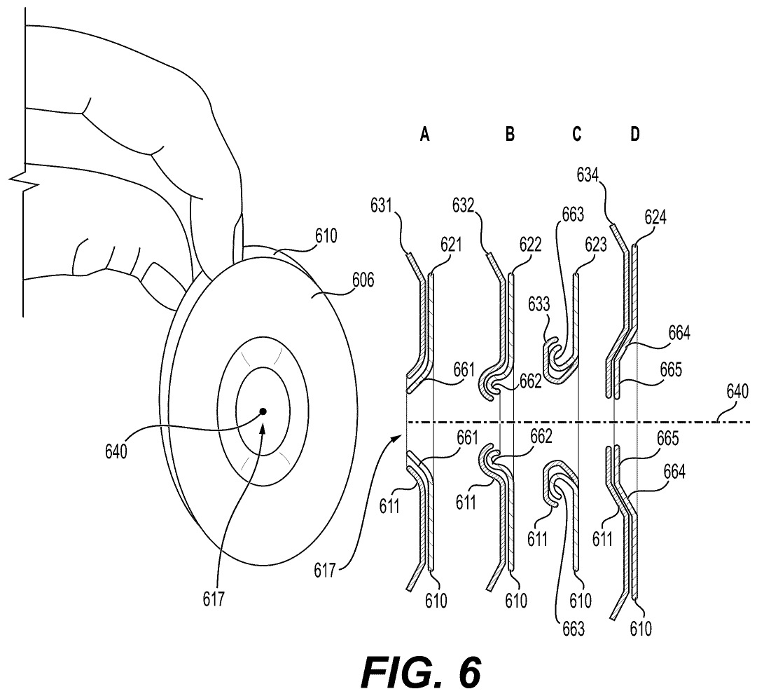

FIG. 6 illustrates various embodiments of wafers 610 that include a convex portion configured to isolate the negative pressure therapy applied by pad 606 from waste contents moving from the patient 101 into bag 115. A central axis 640 of lumen 617 of wafer 610 and pad 606 is shown extending through each example wafer 621-624. Each example wafer 621-624 is shown in a side, cross-sectional view to illustrate the convex portions 661-665 of each example wafer 621-624, and each convex portion 661-665 extends circumferentially completely around central axis 640. Each example wafer 621-624 includes an adhesive portion 611 configured to couple to a wound/resection site of a patient 101 (FIG. 1). First example wafer 621 includes a convex portion 661 that curves outward, and is configured to touch a portion of the patient 101 when wafer 621 and pad 606 are coupled to the patient 101. Second example wafer 622 includes a hook-shaped convex portion 662, and the hook-shaped convex portion 662 curves towards central axis 640. Third example wafer 623 includes a hook-shaped convex portion 663, and the hook-shaped convex portion 663 curves away from central axis 640. Fourth example wafer 624 includes convex portions 664, 665. Convex portions 664 extend outward (towards the patient's tissue). Convex portions 665 extend inward (i.e., towards central axis 640), and are configured to be flush with tissue of patient 101 when wafer 624 and pad 606 are coupled to patient 101. Each of wafers 621-624 are configured to isolate the negative pressure treatment applied by pad 606 to portions of patient 101 outside of the wound/resection opening. Each of wafers 621-624 are configured to prevent suctioning waste and/or other material moving from the patient's digestive tract into bag 115 (FIG. 1). In some examples, any of wafers 621-624 may not include adhesive portion 611.

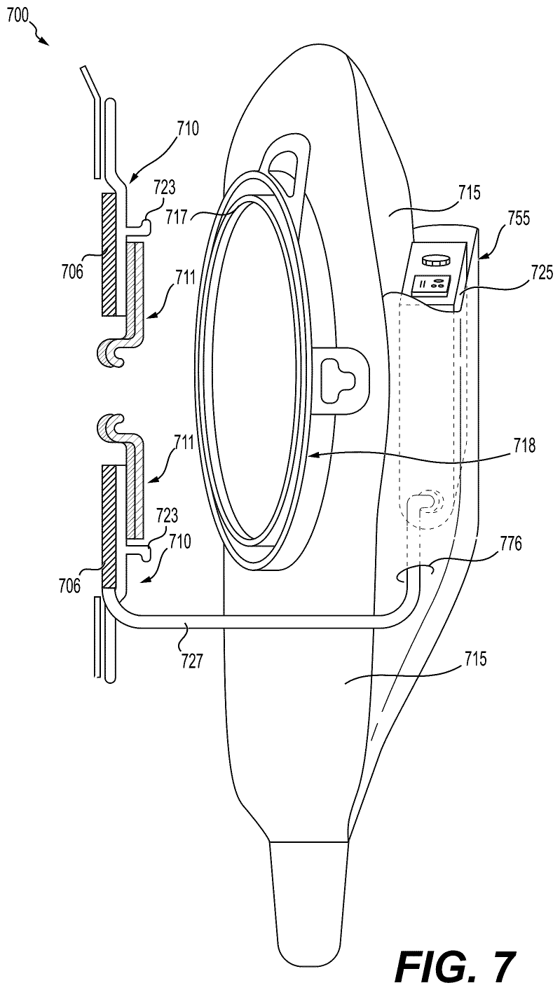

FIG. 7 illustrates an exemplary medical device system 700 including a bag 715, a wafer 710, a pad 706, a tube 727, and a pump assembly 725. Medical device system 700 may have any of the features described herein with regard to medical device system 100. As shown in FIG. 7, bag 715 may include a pocket portion 755 configured to hold pump assembly 725. Pocket portion 755 may include an opening 776 configured to receive tube 727. In some examples, pocket portion 755 may be positioned on an opposite side of bag 715 from distal opening 717 of bag 715. Coupling frame 718 of bag 715 may couple to wafer 710, for example, by engaging ridged portion 723 of wafer 710.

Wafer 710 may include convex portion 711, and convex portion may be interchangeable with other convex portions, such as any of the convex portions illustrated in FIG. 6. Pad 706 may be coupled to wafer 710, and tube 727 may extend to pad 706 to supply negative pressure to pad 706. In some examples, bag 715 may be clear or at least partially transparent to allow a user to see the contents within bag 715. Medical device system 700 may be portable and may allow patient 101 to be mobile while medical device system 700 is coupled to patient 101.

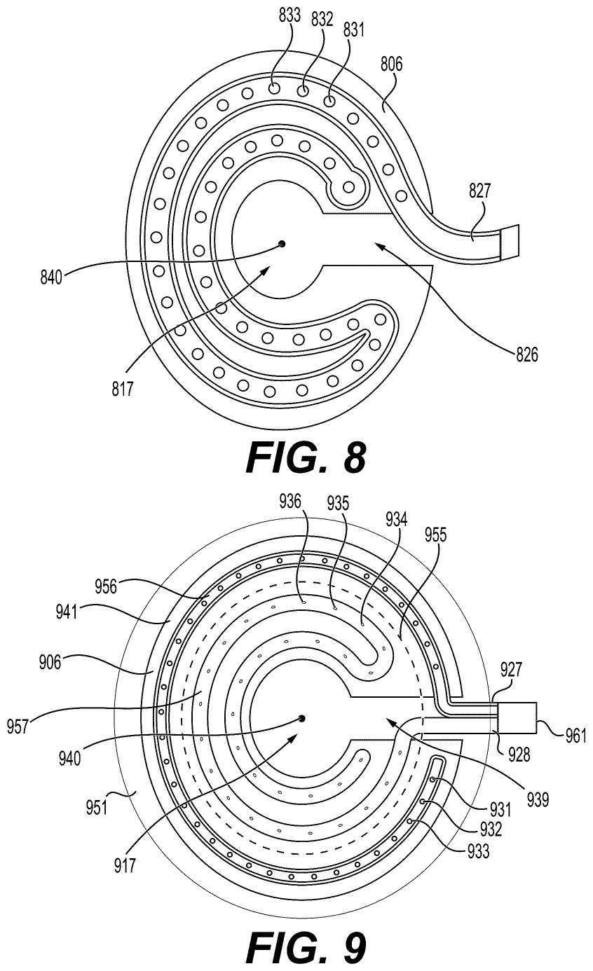

FIG. 8 illustrates an exemplary pad 806 that may be incorporated into any of the medical device systems 100, 700 described herein, and may have any of the features of pads 112, 206, 306, 406, 506, 606, 706 described herein. Pad 806 may be C-shaped, or have the shape of an incomplete disk, to allow pad 806 to be replaced without disassembly of the entire medical device system 100, 700 (e.g., without disassembly of bag 115, etc.). Tube 827 may extend circumferentially around central lumen 817, and openings 831-833 may face a direction away from pad 806 to engage tissue with negative pressure therapy. In other examples, openings 831-833 may face a direction towards pad 806, and may be configured to engage tissue with negative pressure therapy through pad 806 (e.g. through openings/fluid lumens extending through pad 806). A gap 826 may extend from central lumen 817 to a radially-outermost portion of pad 806, relative to central axis 840. To replace pad 806, the vacuum source (e.g., pump assembly 135, 725) may be turned off, and pad 806 may be removed. Then, pad 806 may be replaced with a new C-shaped pad. By providing a C-shaped pad 806, a user may adjust the size of central lumen 817 by overlapping a portion of C-shaped pad (e.g., pulling portions of pad 806 together to eliminate gap 826) to reduce the diameter of central lumen 817, or the ends of the C-shaped pad 806 may be pulled apart to allow for a larger central lumen 817.

Pad 806 may facilitate coupling bag 115 to patient 101, for example, to an ostomy site. Alternatively or additionally, pad 806 may help to enhance bag 115 adhesion to patient 101 and/or allow for less adhesive to be used to couple bag 115 to patient 101. By reducing the amount of adhesion required to couple bag 115 to patient 101, irritation and infection may be reduced at the ostomy site (e.g. wound/resection site).

FIG. 9 illustrates an exemplary pad 906 that may be incorporated into any of the medical device systems 100, 700 described herein, and may have any of the features of pads 112, 206, 306, 406, 506, 606, 706, 806 described herein. Pad 906 may be C-shaped, or have the shape of an incomplete disk, to allow pad 906 to be replaced without disassembly of the entire medical device system 100, 700 (e.g., without disassembly of bag 115, etc.). A first tube 927 and a second tube 928 may be coupled to a first side 941 of pad 906. Each of first tube 927 and second tube 928 may extend circumferentially around central lumen 917 (and a central axis 940). First tube 927 may include openings 931-933 facing a direction away from first side 941, and second tube 928 may include openings 934-936 facing a direction away from first side 941. A connector 961 may receive first tube 927 and second tube 928, and connector 961 may be spaced from a radially-outermost portion, relative to central axis 940, of pad 906. A separation portion 955 may extend circumferentially around central axis 940, and may separate a distal portion 956 of first tube 927 from a distal portion 957 of second tube 928.

Separation portion 955 may be a raised portion of pad 906, may be a divider wall made of plastic or another non-permeable material, may be an adhesive portion, or any other structure configured to fluidically isolate distal portion 956 from distal portion 957. By fluidically isolating distal portion 956 from distal portion 957 when pad 906 is coupled to a patient, first tube 927 may be configured to assist with coupling pad 906 to patient 101 and second tube 928 may be configured to treat a wound site with negative pressure therapy. For example, a higher level of negative pressure may be applied to first tube 927 to facilitate with coupling pad 906 to tissue, and a lower level of negative pressure may be applied to second tube 928 to treat tissue around a wound site.

In some examples, openings 931-933 may be angled to face a direction away from the wound site to help prevent suctioning material from the wound site, and openings 934-936 may be angled to face a direction towards central axis 940 (i.e., towards the wound site) to facilitate collection of material or fluid from the wound site. In some examples, an adhesion patch 951 may be used with pad 906 to facilitate coupling pad 906 to tissue of patient 101. In other examples, adhesion patch 951 may not be used, and pad 906 may be direction coupled to tissue of patient 101. In some examples, first tube 927 may include additional openings 934-936 facing a direction towards wafer 110 to facilitate coupling pad 906 to wafer 110. By providing tube tubes 927, 928 on pad 906, two different treatment zones may be implemented on pad 906 and separated by separation portion 955. In some examples, additional layers of pad material (e.g. porous material, dressing, etc.) may cover first tube 927 and may not cover second tube 928, or vice versa.

Any of the medical device systems 100, 700 described herein may incorporate any of the pads 112, 206, 306, 406, 506, 606, 706, 806, 906 and/or any other feature described in this disclosure.

In any of the above-described embodiments, a medical device system 100, 700 may include one or more components that are metallic, a polymer, machined, formed, stamped, insert molded, or any combination thereof. Any aspect(s) of any one of the above-described embodiments of medical device systems 100, 700, pads 112, 206, 306, 406, 506, 606, 706, 806, 906, or other portions of medical device systems may be incorporated into any of the other medical device system 100, 700 described herein.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device system comprising:
a bag including a first opening;
a pad coupled to the bag proximate to the first opening; and
a tube including a distal portion, wherein the distal portion is coupled to the pad and includes at least one opening;
wherein the tube is configured to provide negative pressure therapy to a wound site.

2. The medical device system of claim 1, further comprising a pump assembly fluidically coupled to the tube, wherein the pump assembly applies negative pressure to the tube.

3. The medical device system of claim 1, wherein the pad is circular and includes a central lumen, and wherein the central lumen aligns with the first opening.

4. The medical device system of claim 3, wherein the distal portion of the tube extends circumferentially around the central lumen.

5. The medical device system of claim 4, wherein the at least one opening of the tube includes a plurality of openings, and wherein the plurality of openings face a direction towards the pad.

6. The medical device system of claim 1, further comprising a filter assembly coupled to the tube and configured to filter material received from the wound site.

7. The medical device system of claim 2, wherein the pump assembly includes a pump and a reservoir configured to receive material from a wound site.

8. The medical device system of claim 1, wherein the pad includes a channel configured to receive the tube, wherein the channel extends circumferentially around a central lumen of the pad.

9. The medical device system of claim 1, wherein the pad includes a perforated portion configured to be removable from the pad.

10. The medical device system of claim 1, wherein the pad includes a first layer of porous material and a second layer of porous material, and wherein a portion of the tube is positioned between the first layer of porous material and the second layer of porous material.

11. The medical device system of claim 1, wherein the pad includes an adhesive on a first side of the pad, and wherein the adhesive extends across a first portion of the first side of the pad and not across a second portion of the first side of the pad.

12. The medical device system of claim 1, further comprising a wafer, wherein a first side of the wafer is configured to couple the bag to the wafer, and wherein a second side of the wafer is configured to couple the pad to the wafer.

13. The medical device system of claim 12, wherein the wafer includes a convex portion, wherein the convex portion extends circumferentially around a central lumen of the wafer, and wherein the tube is positioned radially-outward, relative to a central longitudinal axis of the central lumen, relative to the convex portion.

14. The medical device system of claim 2, wherein the bag includes a pocket and the pump assembly is positioned within the pocket.

15. The medical device system of claim 1, wherein the pad is C-shaped.

16. A medical device system comprising:
a pad configured to couple to tissue, wherein the pad includes a central lumen;
a first tube including a first distal portion, wherein the first distal portion is coupled to the pad, extends circumferentially around the central lumen, and includes at least one opening; and
a second tube including a second distal portion, wherein the second distal portion is coupled to the pad, extends circumferentially around the central lumen, and includes at least one opening;
wherein the first tube is configured to provide negative pressure therapy to a wound site; and
wherein the second tube is configured to facilitate coupling the pad to the tissue.

17. The medical device system of claim 16, wherein the first distal portion includes a plurality of openings facing towards the central lumen, and wherein the second distal portion includes a plurality of openings facing away from the central lumen.

18. The medical device system of claim 17, wherein the first tube is configured to apply a first negative pressure, wherein the second tube is configured to apply a second negative pressure, and wherein the first negative pressure is different from the second negative pressure.

19. The medical device system of claim 18, further comprising an ostomy bag, wherein the pad is coupled to the ostomy bag.

20. A medical device system comprising:
a pad configured to couple to tissue, wherein the pad is a porous material;
a tube including a distal portion, wherein the distal portion is coupled to the pad and includes a plurality of openings facing towards the pad; and
a pump assembly coupled to a proximal portion of the tube;
wherein the pump assembly is configured to provide negative pressure to the tube, the medical device system is configured to provide negative pressure therapy to a wound site, and the tube is positioned within a channel extending circumferentially around a central lumen of the pad.
